(19) United States
(12) Patent Application Publication
(10) Pub. No.: US 2024/0139006 A1
(43) Pub. Date: May 2, 2024

(54) SYSTEMS AND METHODS FOR DEPLOYING STENTS

(71) Applicant: SKW CARDIOVASCULAR TECHNOLOGIES, INC., Escondido, CA (US)

(72) Inventor: Subeer WADIA, Escondido, CA (US)

(21) Appl. No.: 18/281,273

(22) PCT Filed: Mar. 10, 2022
(86) PCT No.: PCT/US2022/019820

§ 371 Date: Sep. 8, 2023

Related U.S. Application Data
(60) Provisional application No. 63/252,587, filed on Oct. 5, 2021.
(60) Provisional application No. 63/159,375, filed on Mar. 10, 2021.

(51) Int. Cl. A61F 2/954; A61F 2/852; A61F 2/958; A61M 25/10; A61F 2/82

(52) U.S. Cl. CPC A61F 2/954; A61F 2/852; A61F 2/958; A61M 25/1002; A61F 2002/821; A61F 2002/826; A61M 2025/1056

(57) ABSTRACT

A system and method for deployment of one or more stents at a bifurcation uses a balloon catheter with distal and proximal guidewire ports that are guided by first and second guidewires. The first and second guidewires are directed into first and second side branches at the bifurcation. The catheter is guided by a combination of the first and second guidewires after inserting the first guidewire through the distal guidewire port and the second guidewire through the proximal guidewire port. The combination of the proximal guidewire port and the second guidewire halts advancement of the catheter and the stent it carries beyond an ostium of the first side branch. Additional stents can be delivered to a second side branch and a main branch using the first and second guidewires without re-positioning of the guidewires.

### CROSS-REFERENCE TO RELATED APPLICATIONS

This application claims the benefit of U.S. Provisional Application No. 63/159,375 filed on Mar. 10, 2021, and Provisional Application No. 63/252,587 filed on Oct. 5, 2021, which are incorporated herein by reference in their entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### FIELD

The present teachings relate to systems and methods for deploying stents in main and side branch vessels.

### INTRODUCTION

Current techniques for stenting bifurcation arterial disease (such as bifurcation coronary artery disease) are difficult. There are currently no commercially available stents designed for bifurcations. The most widely used techniques for treating bifurcation coronary artery disease are: (i) Culotte, (ii) crush/step crush/double-kissing crush techniques, (iii) T and protrusion, or (iv) T stenting. These techniques use currently available technology and are challenging, involving many steps to re-wire branch vessels (sometimes through stent struts), which leads to increased radiation and contrast exposure. In addition, T and protrusion, Culotte, and T stenting do not maintain wire access throughout the bifurcation strategy, leading to the possibility of occluding a vessel during the procedure without easy access. In addition to true bifurcation disease, isolated ostial arterial disease can also be challenging to treat with stents. In the setting of isolated ostial-proximal disease, attempts are made to precisely deploy an ostial stent without missing the ostium (deployment too distally) or hanging the stent into the main vessel (deployment too proximal). However, there is often geographic miss, due to “stent pistoning” during deployment and cardiac motion. The limitations in two dimensional fluoroscopic images also make landing an ostial stent precisely more challenging, requiring multiple views and contrast to precisely identify the origins of branches. A solution is needed to improve treatment of bifurcation disease, including isolated ostial disease, within coronary or peripheral arteries.

### SUMMARY

The present teachings include a system for deploying stents comprising at least one guidewire, a catheter with at least one guidewire port that can accommodate the at least one guidewire, at least one stent with at least one of a slanted edge and a non-slanted edge, and at least one balloon that expands to open the at least one stent. The stents may be advanced into their desired positions by both rapid exchange and over-the-wire techniques. With the former, the catheter only travels along approximately 25 cm of the guidewire before the guidewire is exposed, allowing for easier and quicker advancement and withdrawal of the catheter. With the latter, the guidewire tracks along the entire length of the catheter. The stents may have multiple slanted edges or no slanted edges. In an embodiment, the degree of the slant is between about 30 degrees and about 45 degrees. In another embodiment, the degree of the slant is between about 0 degrees and about 30 degrees. In yet another embodiment, the degree of the slant is between about 45 degrees and about 90 degrees. In yet another embodiment, the angle of the slant is between about 90 degrees and about 120 degrees. In yet another embodiment, the angle of the slant is between about 120 degrees and about 135 degrees. In yet another embodiment, the angle of the slant is between about 135 degrees and about 180 degrees. The guidewire port may accommodate guidewires of various diameters. In an embodiment, the diameter of the guidewire is between about 0.007 inches and about 0.038 inches. In another embodiment, the diameter of the guidewire is between about 0.013 inches and about 0.035 inches. In another embodiment, the diameter of the guidewire is between about 0.020 inches and about 0.030 inches. In addition, the stent may be made of a variety of materials, one of which is a metal (typically cobalt, titanium, or chromium) with a drug coating (drug eluting stents), such as sirolimus or everolimus to prevent tissue ingrowth and restenosis. In an embodiment, the stents are made of at least one of cobalt, titanium, and chromium with a sirolimus drug coating. In another embodiment, the stents are made of at least one of cobalt, titanium, and chromium with an everolimus drug coating. In yet another embodiment, the stents are made of at least one of cobalt, titanium, and chromium with no drug coating. In another embodiment, zotarolimus may be a drug coating for the stents. Zotarolimus, either by itself or in combination with other drug coatings, may be used to prevent smooth muscle proliferation. The diameter of the stent ranges from about 2 mm to about 40 mm. In another embodiment, the diameter of the stent ranges from about 5 mm to about 35 mm. In another embodiment, the diameter of the stent ranges from about 10 mm to about 30 mm. In another embodiment, the diameter of the stent ranges from about 15 mm to about 25 mm. The diameter of the balloon may range from about 2 mm to about 40 mm. In another embodiment, the diameter of the balloon ranges from about 5 mm to about 35 mm. In another embodiment, the diameter of the balloon ranges from about 10 mm to about 30 mm. In another embodiment, the diameter of the balloon ranges from about 15 mm to about 25 mm. The distance between the second guidewire port (proximal guidewire port) and the balloon/stent configuration ranges from about 0 mm to about 20 mm. In other embodiments, the distance between the guidewire port and the balloon/stent configuration ranges from about 2 mm to about 15 mm. In other embodiments, the distance between the guidewire port and the balloon/stent configuration ranges from about 4 mm to about 10 mm. The length of the guidewire lumen ranges from about 0.5 mm to about 400 cm. In other embodiments, the length of the guidewire lumen ranges from about 1 mm to about 200 cm. In other embodiments, the length of the guidewire lumen ranges from about 10 mm to about 100 cm. In other embodiments, the length of the guidewire lumen ranges from about 50 mm to about 50 cm. In other embodiments, the length of the guidewire lumen ranges from about 100 mm to about 10 cm. In other embodiments, the length of the guidewire lumen ranges from about 1 cm to about 5 cm.

In accordance with a further aspect, the system further comprises a radiopaque marker on the at least one stent. The radiopaque marker, especially in relation to the stents in side branch vessels, ensures inflation in the proper orientations of the slanted edge to cover the carina. Orientation can be adjusted by twisting the catheter.

In accordance with yet a further aspect, the at least one stent may be deployed in at least one of a kissing fashion and one at a time. The stents may be delivered at the same time, with one stent going to one side branch vessel, and another stent going to another side branch vessel. At their final destination, when they are expanded by their balloons, the stents are expanded such that their edges are touching (kissing) and extending into the main branch vessel.

In accordance with yet another aspect, the at least one slanted edge provides coverage of a bifurcation carina. By the edge of the stent in side branch vessels having a slanted edge, there can be complete coverage of the bifurcation carina overlapping with the stent in the main branch vessel. There is no need to crush a stent or re-wire through stent struts. In an embodiment, the stents in the side branch vessel may have more than more slanted edge. In another embodiment, the stents in the side bran vessel have only one slanted edge, which is the edge proximally located with the stent in the main branch vessel.

In accordance with yet another aspect, the at least one guidewire port allows delivery of the least one stent with a slanted edge. It is possible for a balloon/stent configuration to be guided to side branch vessels by way of the guidewire port. In an embodiment, the catheter has one guidewire port. In another embodiment, the catheter has more than one guidewire port. More guidewire ports allow for stent deployment in more than one side branch vessel. In another embodiment, multiple guidewire ports also allow for precise advancement of the balloon/stent into one side branch, but not past the ostium, as a second guidewire port will stabilize and anchor the balloon/stent in one side branch blood vessel relative to the second side branch blood vessel. In yet another embodiment, multiple guidewire ports allow a balloon/stent to be advanced over both guidewires to the bifurcation, but preventing the balloon/stent from being advanced into one of the side branch blood vessels. A stent meant for positioning within a side branch vessels move along a guidewire, while a stent for placement in the main branch vessels moves along more than one guidewire. For instance, if there are two guidewire ports, the stent for placement in the main branch vessels moves along two guidewires, and the two stents for placement in two separate side branch vessels move along two separate guidewires.

In accordance with yet another aspect, the at least one guidewire port allows delivery of the least one stent with a non-slanted edge. In an embodiment, a stent with a non-slanted edge may be placed in the side branch vessel, with the non-slanted edge distal from the main branch vessel. In another embodiment, a stent with a non-slanted edge is positioned in the main branch vessel, with the non-slanted edge either distal or proximal to a stent in a side branch vessel.

In accordance with yet another aspect, the slanted edge is at least one of less than 90 degrees and greater than 90 degrees in relation to a length of the at least one stent. The angle of the slant is less than 90 degrees or greater than 90 degrees depending on a side of the stent in which the angle is measured. The slanted edge aids in stent placement, particularly for stents positioned in side branch vessels.

In accordance with yet another aspect, the at least one balloon orients the slanted edges. Twisting the balloon prior to expansion may position stents with slanted edges so that they are deployed correctly. The addition of a second proximal guidewire port orients the slanted edge of the stent upon advancement. Proper orientation also provides maximum coverage of the carina.

In accordance with yet another aspect, the slanted edge is at at least one of a proximal edge of the at least one stent, a distal edge of the at least one stent, and a proximal edge and distal edge of the at least one stent.

In accordance with yet another aspect, the non-slanted edge is at at least one of a proximal edge of the at least one stent, a distal edge of the at least one stent, and a proximal edge and distal edge of the at least one stent. There are various configurations of the stent. The stent may have one slanted edge, or more than one slanted edge. The slanted edge may be the edge that is proximal to the entry point of the stent. The slanted edge may be the edge that is distal to the entry point of the stent. The slanted edge may be both proximal and distal to the entry point of the stent.

In accordance with yet another aspect, at least one balloon has at least one distal end that may accommodate at least one guidewire. The system is capable of accommodating more than one balloon, with each balloon possible of having more than one distal end. Having more than one distal end signifies that the balloon may employ more than one guidewire.

In accordance with yet another aspect, a distal end of the balloon may enter into a side branch vessel as a distal end of a stent abuts the bifurcation carina. In an embodiment, the distal end of the stent stops between about 0 mm and about 0.5 mm from the bifurcation carina. In an embodiment, the distal end of the stent stops between about 0.5 mm and about 1 mm from the bifurcation carina. In another embodiment, the distal end of the stent stops between about 1 mm about 1.5 mm from the bifurcation carina. In another embodiment, the distal end of the stent stops between about 1.5 mm and about 2 mm from the bifurcation carina. In yet other embodiment, the distal end of the stent stops right at the bifurcation carina.

The present teaching also include a method for deploying at least one stent, comprising: providing at least one guidewire, a catheter with at least one guidewire port that can accommodate the at least one guidewire, at least one stent with at least one of a slanted edge and a non-slanted edge, and at least one balloon that expands to open the at least one stent; threading the at least one guidewire through the catheter; moving the at least one balloon encircled by the at least one stent along the catheter to position the at least one stent; and opening the at least one stent by expanding the at least one balloon. Moving a stent to be positioned in a side branch vessel, the stent is guided along a guidewire to the desired position within the side branch vessel. It is possible to guide more than one stent into side branch vessels depending on the number of guidewires. Two stents may be guided into two separate side branch vessels with two guidewires, a guidewire for each stent. In addition, these stents may be deployed one at a time or simultaneously. A stent to be positioned in a main branch vessel moves along the guidewires that were used to position the stents in the side branch vessel. If there are two stents in the side branch vessels (one stent per side branch vessel), the main branch stent moves along two guidewires. Stents with slanted edges are typically placed in the side branch vessels, while those with non-slanted edges remain in the main branch, although it certainly is possible to place slanted edge stents in the main branch and non-slanted edge stents in the side branches.

In accordance with a further aspect, the at least one stent with the slanted edge is positioned in a side blood vessel. Placing a slanted edge stent in a side branch vessel allows for maximal coverage of the carina without crushing of the stent.

In accordance with yet another aspect, the at least one stent with the non-slanted edge is positioned in a main blood vessel. A non-slanted stent in the main branch overlaps with slanted edge stents in side branch vessels. In an embodiment, the side branch stents may have non-slanted edges. In another embodiment, the main branch stents may have slanted edges.

In accordance with yet another aspect, there is overlap between the at least one stent with the slanted edge and the at least one stent with the non-slanted edge. A particular configuration may be side branch stents with slanted edges proximal to a main side stent with non-slanted edges. It is possible for the main branch stent to have a slanted edge, albeit an edge that is not proximal to the slanted edge side branch stents.

In accordance with yet another aspect, the at least one guidewire extends into the side branch blood vessel. Via a guidewire port, a guidewire may extend into as many side branch vessels as there are guidewire ports. For instance, if there are two guidewire ports, there may be a guidewire extending into each side branch vessel.

In accordance with yet another aspect, the slanted edge is at at least one of a proximal edge of the at least one stent, a distal edge of the at least one stent, and a proximal edge and distal edge of the at least one stent. As stents may have several configurations, it is possible to have stents with one slanted edge or more than one slanted edge. With respect to main branch stents and side branch stents, the side branch stents may have a slanted edge that is proximal to the main branch stent. In another embodiment, the side branch stents may also have slanted edge this distal to the main branch stent. In yet another embodiment, the side branch stents may have slanted edges that are proximal and distal to the main branch stent. The slanted edge may be proximal to the entry point of the stent. The slanted edge may be distal to the entry point of the stent. The slanted edge may be proximal and distal to the entry point of the stent (i.e. there are two edges, one is proximal and one is distal to the entry point of the stent).

In accordance with yet another aspect, the non-slanted edge is at at least one of a proximal edge of the at least one stent, a distal edge of the at least one stent, and a proximal edge and distal edge of the at least one stent. In an embodiment, the non-slanted edge may be proximal to the entry point of the stent. In another embodiment, the non-slanted edge may be distal to the entry point of the stent. In yet another embodiment, the non-slanted edge may be both proximal and distal to the entry point of the stent (one edge is proximal and one edge is distal).

In accordance with yet another aspect, the slanted edge is at least one of less than 90 degrees and greater than 90 degrees in relation to a length of the at least one stent. A stent with no slanted edge has an angle of 90 degrees. The slanted edge angles the edge at varying angles, with varying angles being used for specific anatomic variations. The manner in which the angle is determined is based on the interception point of a length and height of the stent or a length and a width of the stent. Whether a slanted edge angle is less than or greater than 90 degrees is dependent on the length of the stent at which the angle is measured.

In accordance with yet another aspect, the at least one balloon orients the slanted edge. Balloon movement, either prior to or during partial expansion, may orient the slanted edge of the stent. Additional guidewires may also assist with orientation of the slanted edges.

In accordance with yet another aspect, a radiopaque marker is on the at least one stent. The radiopaque markers may assist with stent placement. Radiopaque markers may also assist with orientation of the slanted edges.

In accordance with yet another aspect, the balloon used with the stent has at least one distal end, with the distal end capable of entering side branches.

In accordance with yet another aspect, the distal end of the balloon is capable of moving into a side branch vessel once the stent advances right up to the bifurcation carina. A stent may also be mounted on two balloons that are molded together, allowing for Y splitting on proximal ends and distal ends of the balloon. Y splitting may also be on solely the proximal ends of the balloon. Y splitting may also be on solely the distal ends of the balloon.

In accordance with yet another aspect, as the balloon expands the stent, the distal ends also extend any stents that are in the side branches. In an embodiment, the distal ends expand the side branches. In another embodiment, the distal ends expand the side branches and stents within the side branches.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. Depiction of the system showing addition of a second guidewire port proximal to the stent.

FIGS. 2A-C. Depictions of a stent with both slanted edges, a stent with one slanted edge, and stent with non-slanted edges, respectively.

FIG. 3. Depiction of a main branch and side branches of a vessel with two guidewires.

FIG. 4. Depiction of a balloon/stent configuration moving along two guidewires while in a main branch vessel.

FIG. 5. Depiction of a balloon/stent configuration moving along a guidewire while in a side branch vessel, while the second guidewire serves as an anchor point for the balloon/stent, inhibiting further advancement of the balloon/stent more distally into the side branch beyond the bifurcation.

FIG. 6. Depiction of an expanded stent in a side branch vessel deployed at the ostium of the side branch with the balloon delivery catheter removed. In this depiction, the side branch stent has slanted proximal edge which extends into main branch vessel. In another embodiment, side branch stent has non-slanted edge and does not extend into main branch vessel.

FIG. 7. Depiction of two expanded stents, with a second stent deployed at the ostium of another side branch vessel.

FIG. 8. Depiction of a balloon/stent configuration moving over two guidewires simultaneously in the main branch vessel towards the bifurcation (two stents have already been deployed in side branch vessels).

FIG. 9. Depiction of an expanded stent in the main branch vessel overlapping with two expanded stents in side branch vessels.

FIGS. 10A-10B. Depictions of a main branch stent advancing towards a carina and a stent/balloon configuration, respectively.

FIGS. 11A-11B. Depictions of a main branch stent abutting a carina and an enlarged view of the stent/balloon configuration at the carina, respectively.

FIGS. 12A-12B. Depictions of a main branch stent abutting the carina and an enlarge view of the stent/balloon configuration, with the balloon distal ends entering the side branches, respectively.

FIG. 13A-13B. Depictions of the catheter (A) with an enlarged view of the guidewire, balloon, and guidewire port.

### DETAILED DESCRIPTION

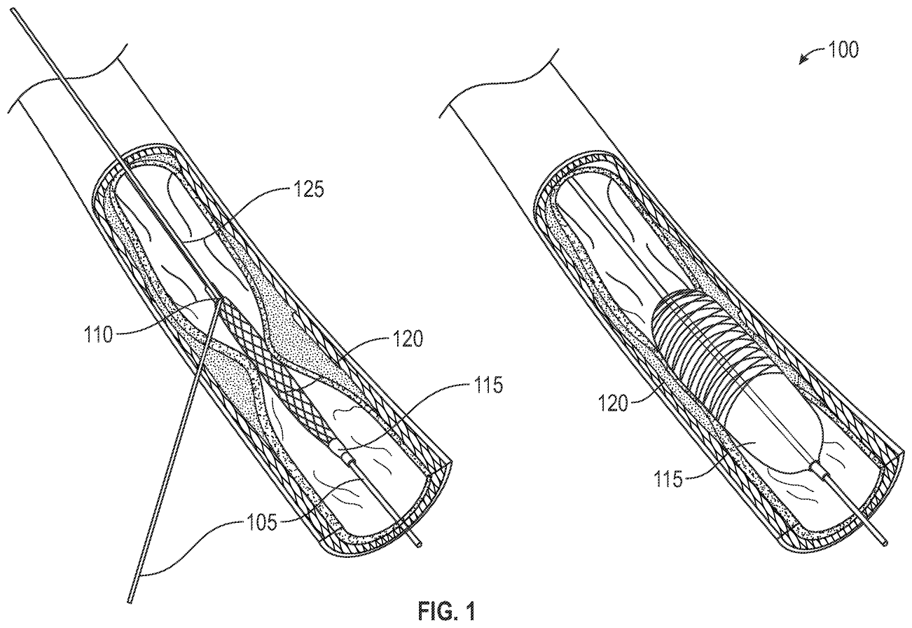

The present invention is directed to a system 100 that deploys stents 120 more precisely by there being at least one guidewire port 110, as shown in FIG. 1. A catheter 125 may have more than one guidewire port 110 for a second guidewire 105 to travel along, making it easier to place stents 120 in blood vessels that branch off from main blood vessels. As a balloon 115 and stent 120 (the balloon 115 is encircled by the stent 120) are guided to their final destination, it is possible for the balloon 115 and stent 120 to stay in the main branch blood vessel or be guided into a side branch blood vessel over guidewires 105. It is possible to have more than one guidewire port 110, so that the balloon 115 and stent 120 may be guided into one side branch blood vessel more precisely to the ostium. Guidewire port 110 serves as an anchor point for the balloon 115 and stent 120, inhibiting further advancement of the balloon 115 and stent 120 over guidewires 105 more distally into the branch vessel.

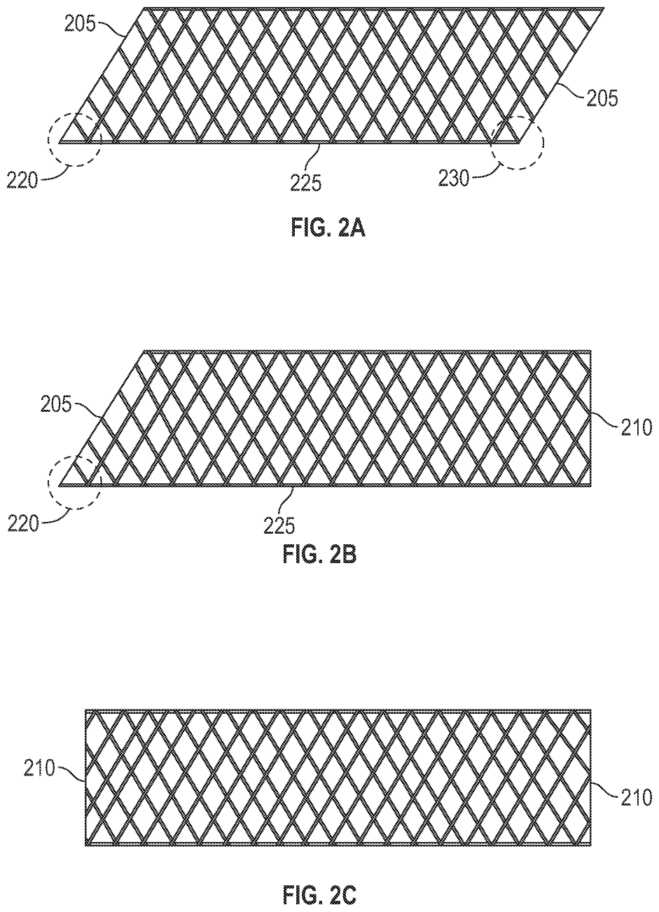

FIGS. 2A-C show various configurations of the stent 120. FIG. 2A depicts a stent 120 with slanted edges 205. FIG. 2B shows a stent 120 with one slanted edge 205 and one non-slanted edge 210. FIG. 2C shows a stent 120 with two non-slanted edges 210. The benefit of a stent 120 having a slanted edge 205, especially in relation to side branch blood vessels, is that the slanted edge 205 may provide complete coverage of the bifurcation carina without the need for crushing a stent 120 or re-wiring through stent struts. The angle of the slant may be less than 90 degrees 220 or greater than 90 degrees 230. Whether the angle is less than 90 degrees or greater than 90 degrees is dependent on the intersection point of the length and height of stent 225. The angle of the stent 225 is measured based on the intersection point of a length of the stent 225 and a height of the stent 225, or the length of the stent 225 and the width of the stent 225. Ultimately, the angle is tailored to a patient's anatomy, with various configurations available to allow selection of an appropriate angle for the unique variations in anatomy.

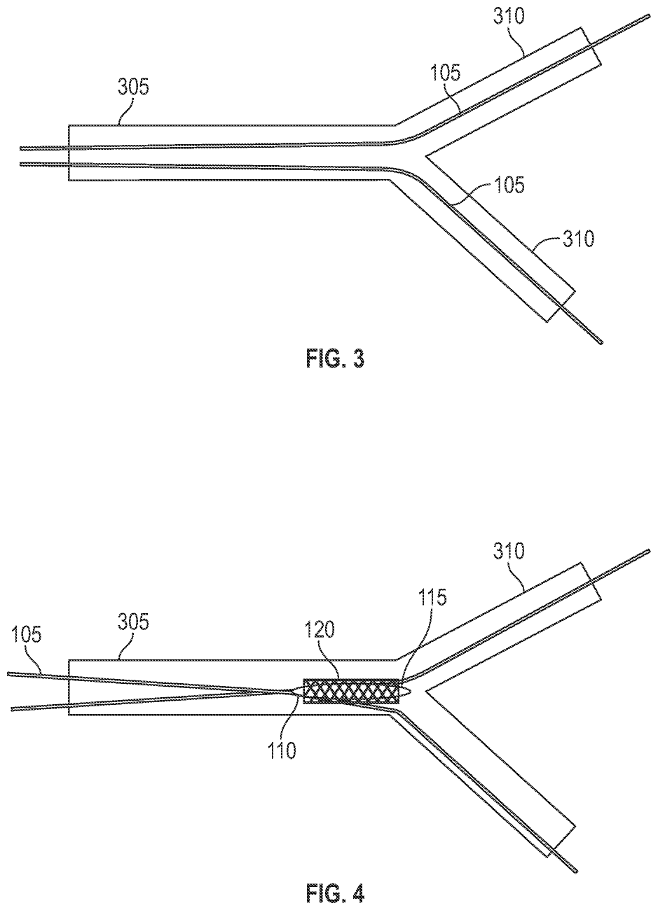

FIG. 3 illustrates a main branch vessel 305 and side branch vessels 310 and the guidewires 105 that will guide a balloon 115 and stent 120 to either stay in the main branch vessel 305 or continue on to the side branch vessels 310.

FIG. 4 shows a configuration of the balloon 115 and stent 120 in the main branch vessel 305. The stent 120 is in the non-expanded state (i.e. the balloon 115 has not been expanded to expand the stent 120). The balloon 115 and stent 120 are advanced over the guidewires 105, with one guidewire 105 threaded through guidewire port 110 on the proximal stent edge. One of the guidewires 105 allows advancement into a side branch vessel, while the second guidewire 105 threaded through guidewire port 110 restricts passage beyond the bifurcation or side branch ostium by way of anchor.

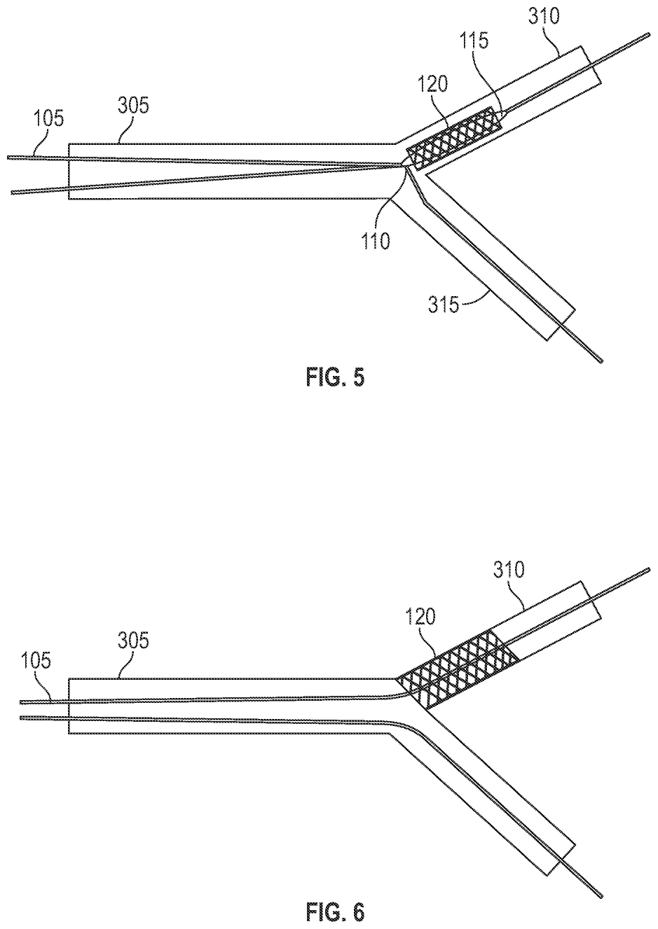

FIG. 5 depicts a balloon 115 and stent 120 being directed into a side branch vessel 310. This is made possible by the catheter's 125 two guidewires 105, one of which is threaded through guidewire exit port 110. The guidewire 105 directs the balloon 115 and stent 120 into a side branch vessel 310, while the guidewire 105 threaded through guidewire port 110 on the proximal balloon/stent edge allows the balloon 115 and stent 120 to be positioned at the ostium, or opening of one side branch 310 relative to another side branch 315.

FIG. 6 shows a stent 120 in the expanded form (i.e. after the balloon 115 is expanded form) while in a side branch vessel 310. The expanded form of the stent is its final form. In an embodiment, the stent 120 has a slanted proximal edge that extends into the main vessel 305. In another embodiment, the stent 120 has a non-slanted proximal edge that does not extend into the main vessel 305.

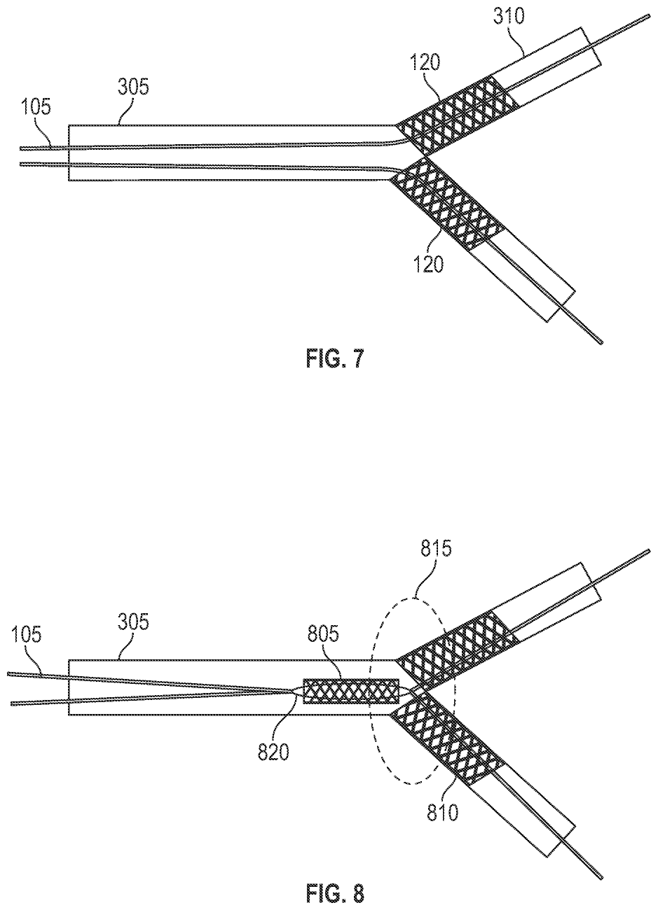

FIG. 7 illustrates two stent 120 in the expanded form in two side branch vessels 310.

In FIG. 8, a non-expanded stent 805 and expanded stent 810 are illustrated. In this instance, a non-expanded stent 805 is in the main branch vessel 305 while two expanded stents 810 are in side branch vessels. The non-expanded stent 805, along with the balloon 820, are being guided towards the expanded stents 810. The non-expanded balloon 820 and stent 805 (together, stent balloon catheter) may accommodate two guidewires 105, which allows the non-expanded stent 805 to travel over both wires 105 but not past the bifurcation 815.

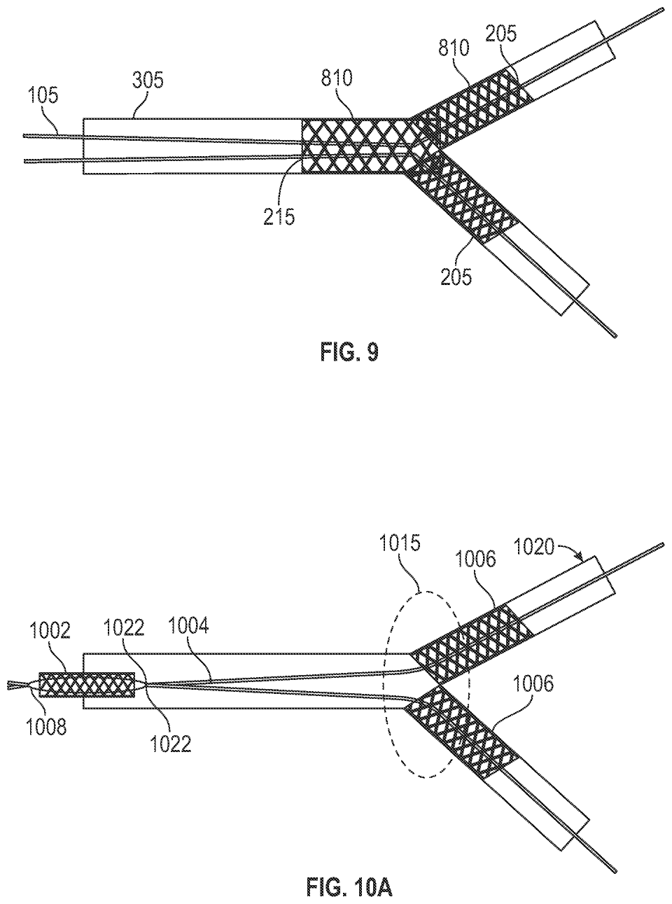

FIG. 9 shows three expanded stents 810, one in the main branch vessel 305 and two in side branch vessel 310. In this embodiment, the expanded stent 810 in the main branch vessel 305 has non-slanted edges 215, while there are stents with slanted edges 205 in the side branch vessels 310. In other embodiments, the type of stent 810 is interchangeable: there may be stents with non-slanted edges 215 and stents with a slanted edge 210 in the side branch vessels 310, and stents with slanted edges 205 210 in the main branch vessel 305. In addition, the stents 810 may have slanted edges 205 proximally and non-slanted edges 215 distally.

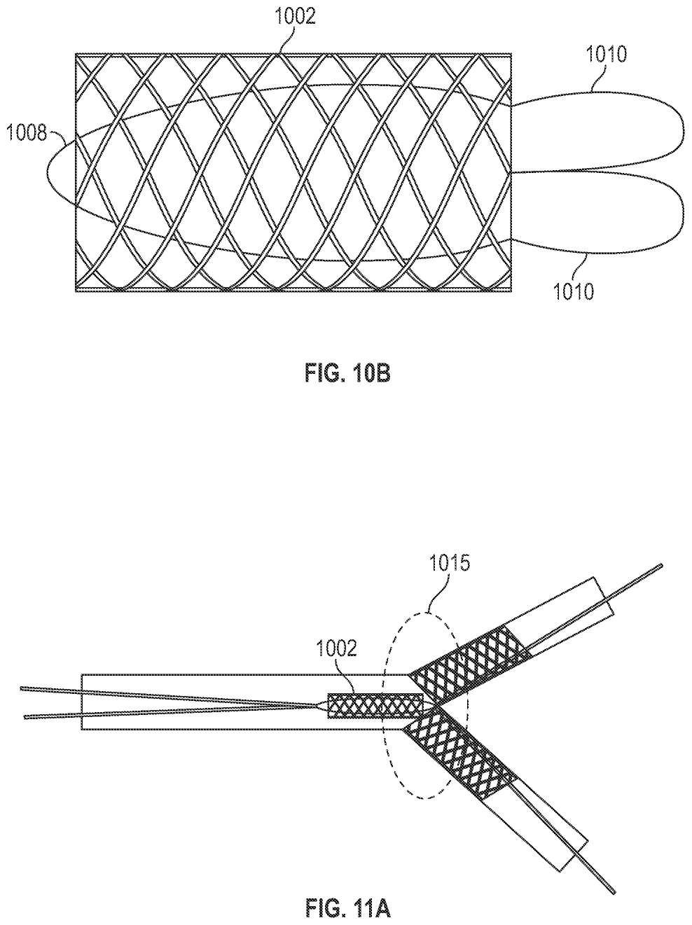

FIG. 10A shows a main branch stent 1002, in a non-expanded state, moving along coronary wires 1004 simultaneously. The stent delivery balloon catheter can accommodate two guidewires. A balloon 1008 is within the main branch stent 1002, with the balloon 1002 inflating once the main branch stent 1002 is at its desired position within the vessel, such as at the carina 1015. Side branch stents 1006 are positioned in the side branches 1020. FIG. 10B shows a balloon 1008 within the main branch stent 1002, with the balloon 1008 having at least one distal end 1010. A distal end 1010 may extend into a side branch 1020 when the main branch stent 1002 abuts the carina 1015. In another embodiment, the balloon 1008 may comprise of two balloons molded together, allowing for a Y-split at the distal end 1010.

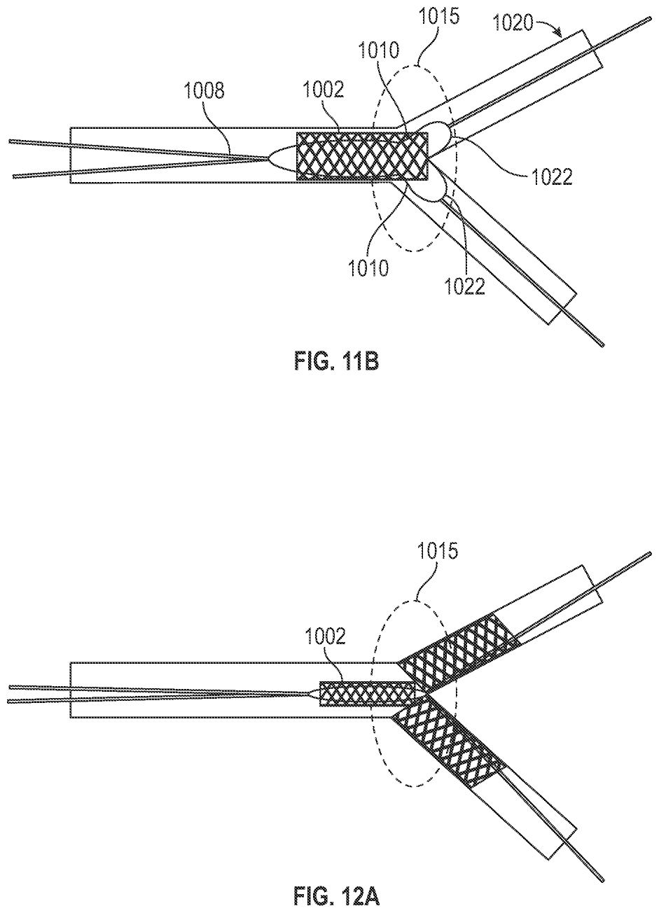

FIG. 11A depicts a main branch stent 1002, with an accompanying balloon 1008 at the carina 1015, right before distal ends 1010 move into the side branch 1020. FIG. 11B shows an enlarged view of FIG. 11A, the distal ends 1010 right at the carina 1015 with the distal ends 1010 about to enter side branches 1020. A balloon 1008 has at least one distal end 1010, making it possible for distal ends 1010 to enter multiple side branches 1020.

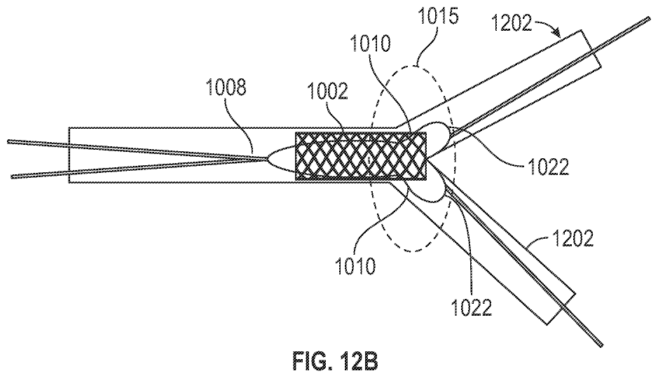

FIG. 12A shows a main branch stent 1002 abutting the carina 1015, with FIG. 12B showing the distal ends 1010 entering the side branches 1020. Once the balloon 1008 is inflated, it is possible for the distal ends 1010 to inflate the side branches 1020 or side branch stents 1006. It is possible to advance side branch stents 1006 along the coronary wires 1004 to their desired positions within side branches 1020, then advance the main branch stent 1002 along the coronary wires 1004 so that the main branch stent 1002 abuts the carina 1005 and balloon 1008. The balloon 1008 with at least one distal end 1010 is enclosed by the main branch stent 1002. Once the main branch stent 1002 abuts the carina 1015 and the distal ends 1010 enter the side branches 1006, the balloon 1008 may be inflated to inflate the main branch stent 1002 and the side branch stents 1006. The guidewire port 1022 shown in FIG. 12B may distal ends 1010 to extend into side branches 1202.

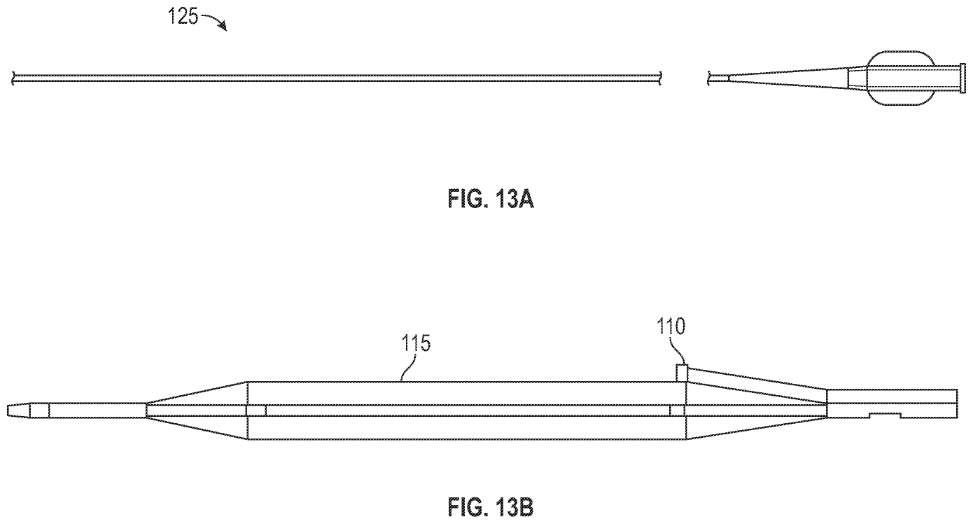

FIG. 13A shows a side view of a stent delivery balloon catheter, with the catheter comprising a hypotube and a shaft. FIG. 13B shows an enlarge view of the stent delivery balloon 115 and guidewire port 110. In addition to a standard guidewire port on the tip of the stent delivery catheter, the addition of a second guidewire port 110 allows for more precise deployment of stents 1006 and balloons 1008. In some embodiments, the guidewire port 110 stabilizes and anchors a stent 1002 1006 and balloon 1008 in a side branch vessel relative to another side branch vessel. In other embodiments, multiple guidewire ports 110 allow a balloon 1008 and stent 1002 1006 to be advanced over multiple guidewires 105 to a bifurcation of blood vessels, all while preventing stent 1006 and balloon 1008 from being advanced into one of the side branch vessels. The guidewire ports 110 may also assist in orienting the slanted edges of stents 1002 1006.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

1-26. (canceled)

27. A system for treatment of vessel having a main branch and at least two side branches, the system comprising:
a first guidewire and a second guidewire configured to be guided into the vessel, each guidewire having a distal end and a proximal end, wherein the distal end of the first guidewire is directed into a first side branch and the distal end of the second guidewire is directed into a second side branch;
a first stent delivery catheter having a balloon, a proximal guidewire port disposed near a proximal end of the balloon, and a distal guidewire port disposed near a distal end of the balloon, wherein the first stent delivery catheter is configured to be guided by a combination of the first guidewire and the second guidewire by inserting the proximal end of the first guidewire through the distal guidewire port and the proximal end of the second guidewire through the proximal guidewire port; and
a first stent having a distal end and a proximal end, the first stent configured to be delivered by the first stent delivery catheter;
wherein the first guidewire is configured to guide the first stent delivery catheter to the first side branch and the proximal guidewire port is configured to cooperate with the second guidewire to halt advancement of the proximal end of the first stent past an ostium of the first side branch, and wherein activation of the balloon deploys the first stent at the ostium of the first side branch.

28. The system of claim 27, wherein the proximal end of the first stent has a slanted edge configured to cover a carina between the at least two side branches and extend into the main branch.

29. The system of claim 27, wherein the proximal end of the first stent has a slanted edge wherein a location of the proximal guidewire port is configured to facilitate orientation of the first stent within the first side branch.

30. The system of claim 27, further comprising:
a second stent delivery catheter having a second balloon, a proximal guidewire port disposed near a proximal end of the second balloon, and a distal guidewire port disposed near a distal end of the second balloon, wherein the second stent delivery catheter is configured to be guided by a combination of the first guidewire and the second guidewire by inserting the proximal end of the second guidewire through the distal guidewire port and the proximal end of the first guidewire through the proximal guidewire port;
a second stent having a distal end and a proximal end, the second stent configured to be delivered by the second stent delivery catheter;
wherein the second guidewire is configured to guide the second stent delivery catheter to the second side branch and the proximal guidewire port is configured to halt advancement of the proximal end of the second stent past an ostium of the second branch, and wherein activation of the second balloon deploys the second stent at the ostium of the second branch.

31. The system of claim 30, further comprising:
a third stent having a distal end and a proximal end, the third stent configured to be delivered by a third stent delivery catheter having a third balloon by inserting the proximal ends of each of the first and second guidewires into a distal guidewire port of the third stent delivery catheter and advancing the third stent to abut the proximal ends of each of the first stent and the second stent, wherein activation of the third balloon deploys the third stent in the main branch.

32. The system of claim 31, wherein the third balloon has a main body with split distal extensions configured to enter the proximal ends of each of the first and second stents.

33. The system of claim 31, wherein the distal end of the third stent and the proximal ends of each of the first and second stents overlap.

34. The system of claim 31, wherein the proximal ends of each of the first and second stents are slanted to ensure coverage of the ostium of each of the first and second side branches and overlap with the distal end of the third stent.

35. The system of claim 27, wherein the first and second guidewires each have a diameter between about 0.007 and 0.038 inch.

36. The system of claim 27, wherein the first and second guidewires each have a diameter between about 0.013 and 0.035 inch.

37. A system for delivery of a stent to a vessel having a main branch and at least two side branches, the system comprising:
a first guidewire and a second guidewire configured to be guided into the vessel, each guidewire having a distal end and a proximal end, wherein the distal end of the first guidewire is directed into a target branch of the at least two side branches and the distal end of the second guidewire is directed into a non-target branch of the at least two side branches; and
a stent delivery catheter having a balloon configured to deploy the stent, a distal guidewire port having an opening near a distal tip of the balloon and a proximal guidewire port disposed near a proximal end of the balloon, the stent delivery catheter configured to be guided by a combination of the first and second guidewires, wherein the proximal end of the first guidewire is inserted through the distal guidewire port and the proximal end of the second guidewire is inserted through the proximal guidewire port, and wherein, as the stent delivery catheter is advanced toward the target branch, a combination of the second guidewire and the guidewire port is configured to halt advancement of a proximal end of the stent past an ostium of the target branch;
wherein activation of the balloon deploys the stent at an ostium of the target branch.

38. The system of claim 37, wherein the proximal end of the stent has a slanted edge configured to cover a carina between the at least two side branches and extend into the main branch.

39. The system of claim 37, wherein the proximal end of the stent has slanted edge wherein a location of the proximal guidewire port is configured to facilitate orientation of the stent within the target branch.

40. The system of claim 37, further comprising a second stent delivery catheter configured to deliver an additional stent to the non-target branch without re-positioning the first and second guidewires by inserting the proximal end of the second guidewire through the distal guidewire port and inserting the proximal end of the first guidewire through the proximal guidewire port.

41. The system of claim 40, further comprising a third stent delivery catheter configured to deliver a main branch stent to abut a carina between the at least two side branches and overlap with the stent and the additional stent by inserting the proximal ends of first and second guidewires through a distal guidewire port of the third stent delivery catheter.

42. The system of claim 41, wherein the third stent delivery catheter comprises one or more modified balloon for delivery of the main branch stent, wherein the one or more modified balloon comprises a main body with split distal extensions configured to enter the proximal ends of each of the stent and the additional stent.

43. A method for delivery of one or more stent to a vessel having a main branch and at least two side branches, the method comprising:
guiding a first guidewire and a second guidewire into the at least two side branches, each guidewire having a distal end and a proximal end, wherein the distal end of the first guidewire is guides into the first side branch and the distal end of the second guidewire is guided into a second side branch;
providing a first stent delivery catheter having a balloon with a first stent releasably disposed thereon, the first stent delivery catheter having a distal guidewire port at a distal tip of the balloon and a proximal guidewire port disposed near a proximal end of the balloon;
inserting the proximal end of the first guidewire through the distal guidewire port and the proximal end of the second guidewire through the proximal guidewire port;
advancing the first stent delivery catheter along a combination of the first and second guidewires until a combination of the proximal guidewire port and the second guidewire halts advancement of a proximal end of the first stent past an ostium of the first side branch;
activating the balloon to deploy the first stent at the ostium of the first side branch; and
withdrawing the first stent delivery catheter from the vessel.

44. The method of claim 43, further comprising:
providing a second stent delivery catheter having a second balloon with a second stent releasably disposed thereon, the second stent delivery catheter having a distal guidewire port at a distal tip of the balloon and a proximal guidewire port disposed near a proximal end of the balloon;
without re-positioning the first and second guidewires, inserting the proximal end of the second guidewire through the distal guidewire port of the second stent delivery catheter and the proximal end of the first guidewire through the proximal guidewire port of the second stent delivery catheter;
advancing the second stent delivery catheter along a combination of the first and second guidewires until the proximal guidewire port halts advancement of a proximal end of the second stent beyond the bifurcation;
activating the second balloon to deploy the second stent at an ostium of the second side branch; and
withdrawing the second stent delivery catheter from the vessel.

45. The method of claim 44, further comprising:
providing a third stent delivery catheter having a third balloon with a third stent releasably disposed thereon, the third stent delivery catheter having a distal guidewire port at a distal tip of the third balloon;
without re-positioning the first and second guidewires, inserting the proximal ends of each of the first and second guidewires through the distal guidewire port;
advancing the third stent delivery catheter along the first and second guidewires until a distal end of the third stent abuts a carina between the first and second side branches;
activating the third balloon to deploy the third stent in the main branch; and
withdrawing the third stent delivery catheter and the first and second guidewires from the vessel.

46. The method of claim 45, wherein the third balloon comprises a modified balloon having a main body with split distal extensions, and wherein advancing the third stent delivery catheter comprises inserting the split distal extensions into the first and second stents.

47. The method of claim 45, wherein the distal end of the third stent and the proximal ends of each of the first and second stents overlap.

48. The method of claim 45, wherein the proximal ends of each of the first and second stents are slanted to ensure coverage of the ostium of each of the first and second side branches.
